(19) United States
(12) Patent Application Publication
(10) Pub. No.: US 2007/0042042 A1
(43) Pub. Date: Feb. 22, 2007

(54) Metformin Tablet with Sustained Release and Method for Preparing the Same

(76) Inventors: Young Gwan Jo, Daejeon (KR); Ja-Seong Koo, Daejeon (KR); Ju-Bin Yim, Daejeon (KR); Yoon-Sik Jun, Seoul (KR)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/428,619

(22) Filed: Jul. 5, 2006

(30) Foreign Application Priority Data
Aug. 18, 2005 (KR) 10-2005-0075923

(51) Int. Cl. A61K 9/22; A61K 31/155

(52) U.S. Cl. 424/468; 264/109; 514/635

(57) ABSTRACT

The present invention relates to a metformin tablet with sustained release and a method for preparing the same, more particularly to an improved metformin tablet with sustained release prepared by manufacturing a composition comprising metformin, which is an active ingredient for treatment of insulin-independent diabetes, and a matrix capable of controlling release rate of metformin into a slug at a given pressure condition, forming a tablet core by dry granulation and then forming a coated film on it, which is slowly released into the body at a constant rate for 24 hours, thereby maintaining a constant blood concentration for 24 hours when administered once a day while offering bioequivalence comparable to that of conventional products, and a method for preparing the same.

### CROSS REFERENCE TO RELATED APPLICATION

This application is based on, and claims priority from Korean Patent Application No. 10-2005-0075923, filed on Aug. 18, 2005, the disclosure of which is hereby incorporated by reference.

### Technical Field

The present invention relates to a metformin tablet with sustained release and a method for preparing the same, more particularly to an improved metformin tablet with sustained release prepared by manufacturing a composition comprising metformin, which is an active ingredient for treatment of insulin-independent diabetes, and a matrix capable of controlling release rate of metformin into a slug at a given pressure condition, forming a tablet core by dry granulation and then forming a coated film on top of it, which is slowly released into the body at a constant rate for 24 hours, thereby maintaining a constant blood concentration for 24 hours when administered once a day while offering bioequivalence comparable to that of conventional products, and a method for preparing the same.

Metformin is a treatment for insulin-independent diabetes used to control blood-sugar level of diabetics. Belonging to a biguanide group, it is highly soluble in water, and can abruptly reduce the sugar level in blood due to rapid release when administered in normal tablets.

In general, the maximum dosage of metformin is 2,550 mg/day. It is administered 2-3 times/day at meals in the amount of 500 or 750 mg in tablet. However, this type of administration may cause abrupt change in the blood concentration of the drug, which may result in adverse reactions and resistance to the drug. Therefore, not only for convenience of patients, but also for efficient treatment, a tablet designed to release the drug content at constant rate for 24 hours is deisrable.

Since metformin hydrochloride is highly soluble in water and hardly permeates the lower GI(gastro intestinal) tract, it is preferable that drugs be absorbed at the upper GI tract.

As described above, metformin has many technical problems and disadvantages to be solved to be developed into a tablet with sustained release. There have been many patents registered worldwide on sustained release tablets of metformin, however, they still have the cost problem since preparation methods thereof are complicated and need several processes.

Retention of drugs with narrow absorption window like metformin hydrochloride in the GI duct needs to be prolonged by swelling and a commercially available sustained release system is required.

But, the osmotic release formulation using semipermeable coating, the controlled release formulation using enteric coating and the controlled release formulation utilizing controlled granular dissolution rate are not suitable for metformin considering its narrow absorption window. Moreover, they require expensive equipments for preparation.

U.S. Pat. No. 5,955,106 discloses a pharmaceutical composition comprising metformin hydrochloride and having a residual moisture content of about 0.5-3% by weight. The relatively low moisture content resolves the capping problem of the tablet. The above patent uses a retarding agent selected from the group consisting of cellulose derivatives, dextrins, starch, carbohydrate-based polymers, natural gums, xanthane gum, alginates, gelatin, polyacrylic acid, polyvinyl alcohol and polyvinylpyrrolidone.

However, because of the relatively large amount of metformin for unit dose, the tablet or capsule requires a large volume. Also, since metformin is highly soluble, use of a relatively large amount of polymers is inevitable, which makes intake of the resultant large-sized oral formulation difficult. Further, the compressibility problem of metformin still remains to be solved.

The controlled release hydrophilic drugs of Depomed [PCT/US1998/11302] is less effective than the 24-hour controlled release of the present invention since release of active ingredients are completed, basically, within 8 hours. The application does not specify construction or design of materials that do not allow controlled release. For such drugs that are to be contained in large amount for unit dose and are hardly compressible, as metformin, controlled release is impossible with general polymers. Even if they are prepared into tablets, they tend to be too large for oral administration.

Andrex Co., Ltd. has disclosed a method of forming a semipermeable coat on a pharmaceutical composition followed by penetrating the coat with a laser drill [PCT/US1999/06024]. This method is disadvantageous in that the laser drill is expensive and the pores through which the drug is released may have different size depending upon the operator or processing conditions. Thus, it is not desirable for diabetic treatment and is also not economical.

In U.S. Patent Application No. 2004/0161461, Sethpawan discloses a method of dissolving a binder in a solvent, granulating by adding a swelling agent, drying and converting the granules into a tablet core and coating a semipermeable film on it. However, this method is limited in that uniform coating cannot be achieved due to its rather complex coating process.

In U.S. Patent Application No. 2004/0109891, Sanghri and Pradeep propose introducing natural gums like xanthane gum and locust bean gum to metformin salt. However, this technique is not so efficient because calcium sulfate, or gypsum, used as ionizing agent is insoluble in water, thereby being unable to form a gel.

In U.S. Pat. No. 6,682,759, Jong C Lim and John N. Shell present a two-phase controlled release technique of coating a fast-release coat on a sustained-release core. The sustained-release core can be prepared with uniform quality without difficulty. However, it is difficult to form a uniform fast-release coat since it has to be prepared by wet coating. Moreover, it has the stability problem of reduced activity and is disadvantageous in offering equivalency of fast-release drugs.

In U.S. Patent Application No. 2004/0076667, Kumar Gidwani et al. propose a method of melting fatty acid and fatty acid ester at high temperature and granulating thereby manufacturing the same into a tablet. In this method, the drug may be decomposed at high temperature and the related process is very complicated.

In U.S. Patent Application No. 2004/0086566, Zhang and Xiaoying disclose a method, which is similar to that of Kumar Gidwani, of mixing metformin with wax and forming a tablet by hot melt process.

In U.S. Pat. No. 6,676,966, Amina Odidi and Isa Odidi disclose a sustained release tablet in which a methacrylic acid copolymer is used as coating film. The coat of the resultant formulation is dissolved not at acidic pH but at pH 5-6 or above. To put it another way, metformin is absorbed not at acidic pH but at weekly acidic pH of 5-6. Thus, it is not absorbed in the upper GI tract.

### DISCLOSURE OF THE INVENTION

The present inventors have made various efforts to solve these problems, and as a result, they have discovered that a sustained release drug system comprising metformin as an active ingredient and a matrix for controlling the release rate of metformin has prolonged retention time in the GI duct by the swelling mechanism and that a composition comprising metformin and a matrix can be prepared into a slug by applying pressure, be granulated and prepared into a tablet with relative easiness.

As presented by the present invention, slug formation under a predetermined pressure condition solves the problem of almost impossible tablet production by the dry method caused by poor compressibility and fluidity of metformin and metformin hydrochloride. The metformin tablet with sustained release of the present invention solves the size problem of the tablet, providing convenience in patients' intake of the drug.

Accordingly, in an aspect of the present invention, there is provided a method for an improved metformin tablet with sustained release enabling sustained release of metformin, offering simple producing method as to be applied in a commercial scale and making intake more convenient with reduced size and a preparation method thereof.

### BRIEF DESCRIPTION OF THE DRAWING

The above and other objects, features and other advantages of the present invention will be more clearly understood from the following detailed description taken in conjunction with the accompanying drawing, in which;

FIG. 1 is a graph that compares the dissolution rate of the metformin tablet with sustained release prepared in Example 1 with that of the glucophage tablet with sustained release available in the market.

FIG. 2 is a graph that compares the bioequivalence of the metformin tablet with sustained release prepared in Example 1 with that of the glucophage tablet with sustained release available in the market.

### BEST MODE FOR CARRYING OUT THE INVENTION

The present invention relates to a method for preparing a metformin tablet with sustained release comprising converting a pharmaceutical composition comprising metformin or a pharmaceutically acceptable salt thereof as an active ingredient and a matrix into a slug at a pressure of 5-30 MPa, granulating the slug to particles with a size of 12-30 meshes, converting the granule into a tablet core and forming a coated film on the tablet core.

The present invention relates to a metformin tablet with sustained release prepared by the above method which comprises a single-phase tablet core comprising metformin or a pharmaceutically acceptable salt thereof as an active ingredient and a matrix and a coated film covering outer surface of the tablet core.

Hereunder is given a detailed description of the present invention.

The present invention relates to a metformin tablet with sustained release and a method for preparing the same, more particularly to an improved metformin tablet with sustained release prepared by manufacturing a composition comprising metformin, which is an active ingredient for treatment of insulin-independent diabetes, and a matrix capable of controlling release rate of metformin into a slug at a given pressure condition, forming a tablet core by dry granulation and then forming a coated film on it, which is slowly released into the body at a constant rate for 24 hours, thereby maintaining a constant blood concentration for 24 hours when administered once a day while offering bioequivalence comparable to that of conventional products, and a method for preparing the same.

The present invention provides the optimum administration formulation for treatment of insulin-independent diabetes maintaining sustained intake of metformin which has high solubility in water and narrow having absorption window in the upper GI duct and has to be comprised in a large amount for unit dose.

Description on each step of the method for preparing a metformin tablet with sustained release in accordance with the present invention is given hereinbelow.

In the first step, a pharmaceutical composition comprising metformin or a pharmaceutically acceptable salt thereof as an active ingredient and a matrix is made into a slug at a pressure of 5-30 MPa.

As an active ingredient, metformin or a pharmaceutically acceptable salt thereof, most preferably metformin hydrochloride, is used. This description mainly describes the use of metformin hydrochloride, but the scope of the present invention is not limited thereto.

Metformin is contained in the amount of 25-75 wt %, preferably in 30-70 wt %, and most preferably in 35-65 wt %, based on the total weight of the tablet.

When the tablet is taken, the matrix swells, so that the metformin remains longer in the GI duct, thereby controlling absorption of metformin. As a matrix, at least one selected from the group consisting of cellulose derivatives, dextrin, starch, carbohydrate-based polymers, natural gums, guar gum, tragacanth, acacia gum, locust bean gum, xanthane gum, alginates, gelatin, polyacrylic acid, polyvinyl alcohol, polyvinylpyrrolidone, polyvinyl acetate and methacrylate copolymer derivatives or a mixture thereof may be used. The matrix is contained in the amount of 25-75 wt %, preferably in 30-70 wt %, and most preferably in 35-65 wt %, based on the total weight of the tablet. If the content of the matrix is below 25 wt %, the drug is released too fast. In contrast, if it exceeds 75 wt %, the drug is released very slowly and the tablet becomes too large, thus making it difficult to administer.

The slug means an aggregate prepared by strongly compressing an active ingredient with pharmaceutical additives. The slug preparation of the present invention increases density of the granule and improves fluidity, thereby reducing volume of the tablet. One of the technical features of the present invention is the pressure condition in preparing the pharmaceutical composition comprising metformin or a pharmaceutically acceptable salt thereof and a matrix into a slug. The slug preparation is performed at 5-30 MPa, preferably at 10-25 MPa, and more preferably at 15-20 MPa. If the pressure is below 5 MPa, granulation is insufficient, so that wanted compressibility and fluidity cannot be attained. In contrast, if it exceeds 30 MPa, the slug becomes too rigid, and thus it takes long time for the slug preparation and granulation.

In the second step, the slug is granulated into particles with a size of 12-30 meshes and formed into a tablet core.

When the slug formed in the first step is granulated, it improves density, fluidity and compressibility. The granulation is performed to particles with a size of 12-30 meshes, preferably to 14-24 meshes, and most preferably to 16-20 meshes. If the particle size is smaller than 30 meshes, desired particle density and fluidity cannot be attained. In contrast, if it exceeds 12 meshes, compressibility of the tablet becomes poor.

A single-phase tablet core is obtained following the first and second steps. The resultant tablet core has improved compressibility and fluidity because metformin hydrochloride, an active ingredient, strongly binds to the matrix, a polymer, by strong pressure. As a result, limitation in dry grinding caused by high solubility of metformin in water can be solved.

Conventionally, a large amount of matrix had to be used to offer sustained controlled release since metformin is highly soluble in water, which increased the volume of the tablet and made it difficult to take.

The metformin tablet with sustained release prepared in accordance with the present invention has a volume reduced by 10-20%. Thus, patients can take tablets more conveniently and consistent treatment becomes possible.

In addition to the active ingredient and the matrix, an additive selected from pharmaceutically acceptable diluents (starch, microcrystalline cellulose, lactose, glucose, mannitol, alginate, alkaline earth metal salts, clay, polyethylene glycol, dicalcium phosphate, etc.), binders (starch, microcrystalline cellulose, high dispersible silica, mannitol, lactose, polyethylene glycol, polyvinylpyrrolidone, crosslinked polyvinylpyrrolidone, crosslinked carboxymethylcellulose, hydroxypropylmethylcellulose, hydroxypropylcellulose, natural gums, synthetic gums, copovidone, gelatin, etc.), disintegrators (starch or modified starches, particularly sodium starch glycolate, cornstarch, potato starch or pre-gelatinated starch, clays, particularly bentonite, montmorillonite or veegum; celluloses, particularly microcrystalline cellulose like hydroxypropylcellulose or carboxymethylcellulose; alginates, particularly sodium alginate or alginic acid; crosslinked celluloses, particularly croscarmellose sodium; gums, particularly guar gum or xanthane gum; crosslinked polymers, particularly crospovidone; foaming agents, particularly sodium bicarbonate or citric acid; or a mixture thereof), lubricants (talc, sodium stearate, stearates of alkaline earth metals, such as calcium and zinc, lauryl sulfate, hydrogenated plant oil, sodium benzoate, sodium stearylfumarate, glyceryl monostearate, polyethylene glycol 4000, etc.), colorants and perfumes can be included in the tablet core, as long as the effect of the present invention is not hindered.

In the examples to be described below, microcrystalline cellulose, Ludipress® (BASF, Germany), Aerosil 200 (Degussa, Germany), sodium stearate, etc. were used for the additive. However, the scope of the present invention is not limited to those examples. An additional amount of the additive may be selected by the one skilled in the art.

In the third step, a coated film is formed on the surface of the tablet core.

The coated film formed on the surface of the tablet core is a mixture between at least one selected from the group consisting of a coating agent selected from the group consisting of cellulose derivatives, sugar derivatives, polyvinyl derivatives, waxes, fats and gelatin and between at least one supplementary agent selected from the group consisting of polyethylene glycol, ethylcellulose, titanium oxide and diethyl phthalate. The coated film may account for 0.5-15 wt %, preferably 1-10 wt %, most preferably 2-5 wt % of the total weight of the tablet. If the content of the coated film is below 0.5 wt %, content of metformin tends to be low. In contrast, if it exceeds 15 wt %, absorption in the upper GI duct may become difficult because it takes too long for disintegration.

The coated film may be formed by a method selected by the one skilled in the art. For example, fluid bed coating, pan coating, etc. may be used, and preferably pan coating.

The coated film may be further coated to secure stability of the active ingredient.

As described above, the tablet prepared by manufacturing a composition comprising metformin and a matrix into a slug under a predetermined pressure, forming the slug into a tablet core by dry granulation and forming a coated film on the tablet core has such a superior dissolution property that it can be slowly released into the body at coistant rate for 24 hours. Thus, it can offer constant blood level of the drug for 24 hours with one administration a day while offering bioequivalence comparable to that of conventional tablets.

Hereinafter, the present invention is described in further detail through examples. However, the following examples are only for the understanding of the invention and the invention should not be construed as limiting the scope of the invention.

### EXAMPLE 1

### Preparation of Tablet Containing 500 mg of Metformin

Metformin hydrochloride, hydroxypropyl methylcellulose and light anhydrous silicic acid were mixed as shown in Table 1 below. The mixture was compacted with a roller at a pressure of 16-17 MPa into a slug. The slug was sieved with a 14-mesh sieve, mixed with sodium stearate and then prepared into a tablet core. A coated film was formed on the tablet with Opadry OY-C-7000A core using Hi-Coater (SFC-30N, Sejong Machinery, Korea) to obtain a metformin tablet with sustained release (Metformin XR tablet 500 mg) containing 500 mg of metformin.

### EXAMPLE 2

### Preparation of Tablet Containing 500 mg of Metformin

Metformin hydrochloride, sodium carboxymethylcellulose, Avicel PH101 and light anhydrous were mixed as shown in Table 1. A metformin tablet with sustained release (Metformin XR tablet 500 mg) containing 500 mg of metformin was prepared same as in Example 1.

### EXAMPLE 3

### Preparation of Tablet Containing 500 mg of Metformin

Metformin hydrochloride, guar gum and light anhydrous were mixed as shown in Table 1. A metformin tablet with sustained release (Metformin XR tablet 500 mg) containing 500 mg of metformin was prepared same as in Example 1.

### EXAMPLE 4

### Preparation of Tablet Containing 750 mg of Metformin

Metformin hydrochloride, hydroxypropyl methylcellulose and light anhydrous were mixed as shown in Table 1. A metformin tablet with sustained release (Metformin XR tablet 750 mg) containing 750 mg of metformin was prepared same as in Example 1.

### COMPARATIVE EXAMPLE 1

### Preparation of Tablet Containing 500 mg of Metformin

Metformin hydrochloride, hydroxypropyl methylcellulose, light anhydrous silicic acid and sodium stearate were mixed as shown in Table 1. The mixture was directly compressed to obtain a tablet core. A coated film was formed on the tablet core with Opadry OY-C-7000A using Hi-Coater (SFC-30N, Sejong Machinery, Korea) to obtain a metformin tablet with sustained release (Metformin XR tablet 500 mg) containing 500 mg of metformin.

### COMPARATIVE EXAMPLE 2

### Preparation of Tablet Containing 500 mg of Metformin

Metformin hydrochloride, hydroxypropyl methylcellulose, light anhydrous silicic acid and sodium stearate were mixed as shown in Table 1. A metformin tablet with sustained release (Metformin XR tablet 750 mg) containing 750 mg of metformin was prepared same as in Comparative Example 1.

TABLE 1

| | Composition (mg/tablet) | | | | | |
|---|---|---|---|---|---|---|
| | | | | | Comp. | Comp. |
| Constituents | Ex. 1 | Ex. 2 | Ex. 3 | Ex. 4 | Ex. 1 | Ex. 2 |
| Metformin | 500 | 500 | 500 | 750 | 500 | 750 |
| hydrochloride | | | | | | |
| Hydroxypropyl | 500 | — | — | 500 | 500 | 500 |
| methylcellulose¹⁾ | | | | | | |
| Sodium carboxy- | — | 450 | — | — | — | — |
| methylcellulose²⁾ | | | | | | |
| Guar gum | — | — | 500 | — | — | — |
| Avicel PH101³⁾ | — | 41 | — | — | — | — |
| Aerosil 200⁴⁾ | 5 | 5 | 5 | 7.5 | 5 | 7.5 |
| Magnesium | 5 | 4 | 4 | 7.5 | 5 | 7.5 |
| stearate | | | | | | |
| Opadry | 40 | 40 | 40 | 60 | 40 | 60 |
| OY-C-7000A⁵⁾ | | | | | | |
| Total | 1050 | 1040 | 1040 | 1325 | 1050 | 1325 |

¹⁾Dow Chemical, USA
²⁾Borak, Korea
³⁾Asahi, Japan
⁴⁾Degussa, Germany
⁵⁾ColorCone, USA

### EXPERIMENTAL EXAMPLE 1

### Comparative Physical Property Test

The source materials of Examples 1 and 4 were mixed and made into slugs at a strong pressure of 16-17 MPa and then granulated into semiproducts Compressibility and fluidity of the semiproducts were compared with those of Comparative Examples 1 and 2 as follows.

Tapped density and fluidity were measured to compare physical properties of the semiproducts before and after roller compacting. Volume per weight of the tablets prepared from the semiproducts was compared.

Tapped density was measured with Tapped Volumeter SVM102 of ERWEKA and fluidity was measured with Granulate Tester GT-L of ERWEKA.

TABLE 2

| | Tapped density | Fluidity | Tablet volume |
|---|---|---|---|
| Composition | (mg/ml) | (g/s) | (ml/20 tablets) |
| Ex. 1 | 0.69 | 10.8 | 15.7 |
| Comp. Ex. 1 | 0.61 | 6.1 | 18.0 |
| Ex. 4 | 0.72 | 9.6 | 22.2 |
| Comp. Ex. 2 | 0.64 | 6.3 | 25.5 |

As seen in Table 2, the dry granules prepared from the slugs (Examples 1 and 4) had very superior fluidity and compressibility to those prepared without a slugging step (Comparative Examples 1 and 2). Also, they had reduced volume per unit weight.

Therefore, the tablet according to the present invention is an ideal controlled release formulation since it can be produced in commercial scale simply by dry granulation without complicated or expensive processes.

Also, it is convenient to take because the tablet has 10-20% reduced volume.

### EXPERIMENTAL EXAMPLE 2

### Comparative Dissolution Profile Test

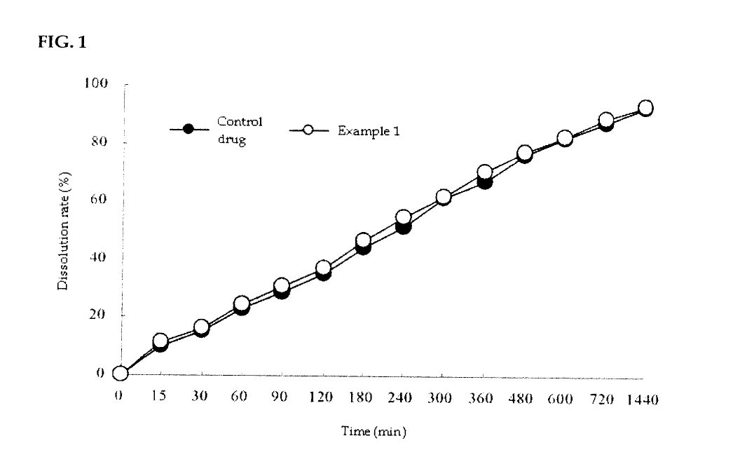

Dissolution profile of the metformin tablet with sustained release of the present invention (Example 1) was compared with that of a commercially available control drug (Glucophage XL of BMS, USA). The paddle method was used to determine the dissolution property. The result is shown in FIG. 1.

As seen in FIG. 1, the metformin tablet with sustained release of the present invention showed dissolution property comparable to that of the control drug. While the control drug is a two-phase sustained release tablet prepared by a complicated process (Korean Patent Application No. 2000-7010280), the tablet of the present invention shows comparable dissolution property although it is prepared by a simple process of dry granulation.

### EXPERIMENTAL EXAMPLE 3

### Bioequivalence Test

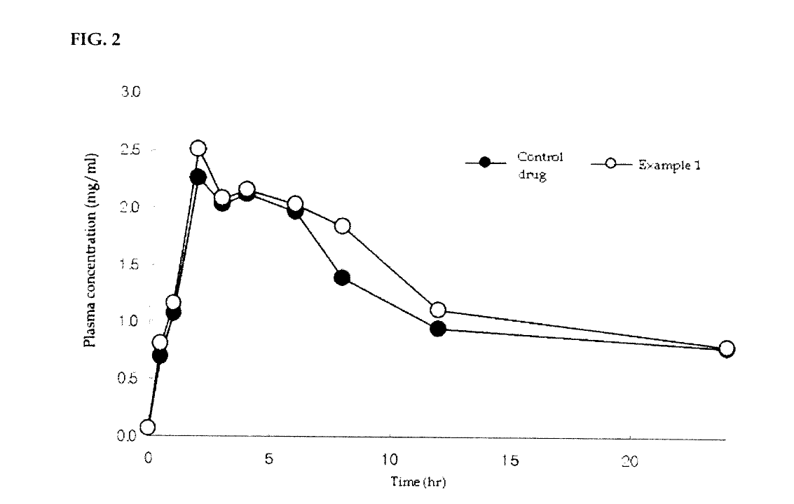

Bioequivalence of the metformin tablet with sustained release of the present invention (Example 1) was compared with that of a commercially available control drug (Glucophage XL of BMS, USA). The result is shown in FIG. 2 and Table 3 below.

As seen in FIG. 2, the metformin tablet with sustained release of the present invention showed bioequivalence comparable to that of the control drug.

TABLE 3

| | Cₘₐₓ (μg/ml) | AUC (μg · hr/ml) |
|---|---|---|
| Example 1 | 2.4078 | 45.1619 |
| Control drug | 2.2517 | 43.7521 |

### INDUSTRIAL APPLICABILITY

As apparent from the above description, the present invention is effective in producing a metformin tablet with sustained release in commercial scale by relatively simple dry granulation of preparing an active ingredient and a polymer into a slug at a predetermined pressure of 5-30 MPa, granulating the slug and converting it into a tablet.

The metformin tablet with sustained release of the present invention, which comprises a single-phase tablet core and a coated film, secures drug stability and enables sustained drug release. That is, since the drug is slowly related for 24 hours at a constant rate in the body, a constant blood level can be attained for 24 hours with one administration a day. Also, the tablet offers good bioequivalence.

As metformin needs a large amount for unit dose and it requires a large amount of polymers to offer wanted sustained release because it is highly soluble in water, the metformin tablet tends to have a large volume. The present invention solves this problem through dry granulation and offers a tablet having a reduced volume for patients' convenience in administration of the drug.

While the present invention has been described in detail with reference to the preferred embodiments, those skilled in the art will appreciate that various modifications and substitutions can be made thereto without departing from the spirit and scope of the present invention as set forth in the appended claims.

1. A method for preparing a metformin tablet with sustained release comprising:
(a) converting a pharmaceutical composition comprising metformin or a pharmaceutically acceptable salt thereof as an active ingredient and a matrix into a slug at a pressure of 5-30 MPa;
(b) granulating said slug into particles with a size of 12-30 meshes and preparing them into a tablet core; and
(c) forming a coated film on the surface of the tablet core.

2. The method of claim 1, wherein said pharmaceutically acceptable salt of metformin is an added salt of an inorganic or organic acid.

3. The method of claim 1, wherein said pharmaceutically acceptable salt of metformin is metformin hydrochloride.

4. The method of claim 1, wherein said metformin or said pharmaceutically acceptable salt is contained in the amount of 25-75 wt % based on the total weight of the tablet.

5. The method of claim 1, wherein the matrix is at least one polymer selected from the group consisting of cellulose derivatives, dextrin, starch, carbohydrate-based polymers, natural gums, guar gum, tragacanth, acacia gum, locust bean gum, xanthane gum, alginates, gelatin, polyacrylic acid, polyvinyl alcohol, polyvinylpyrrolidone, polyvinyl acetate and methacrylate copolymer derivatives and a mixture thereof.

6. The method of claim 1, wherein the matrix is contained in the amount of 25-75 wt % based on the total weight of the tablet.

7. The method of claim 1, wherein said coated film comprises a mixture between at least one of a coating agent selected from the group consisting of cellulose derivatives, sugar derivatives, polyvinyl derivatives, waxes, fats and gelatin and at least one of a supplementary agent selected from the group consisting of polyethylene glycol, ethylcellulose, titanium oxide and diethyl phthalate.

8. The method of claim 1, wherein said coated film is contained in the amount of 0.5-15 wt % based on the total weight of the tablet.

9. A metformin tablet with sustained release prepared by a method of claims 1, which comprises a single-phase tablet core comprising metformin or a pharmaceutically acceptable salt thereof as an active ingredient and a matrix and a coated film covering the outer surface of said tablet core.
